(19) United States
(12) Patent Application Publication
(10) Pub. No.: US 2009/0228019 A1
(43) Pub. Date: Sep. 10, 2009

(54) ROBOTIC SURGICAL SYSTEM

(76) Inventors: Yosef GROSS, Moshav Mazor (IL); Nir Lilach, Kfar Yehoshua (IL); Eliahu Eliachar, Haifa (IL); Ofer Yossepowitch, Petach Tikvah (IL); Dan Sade Hochstadter, Kibbutz Bet Alfa (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/045,230

(22) Filed: Mar. 10, 2008

(51) Int. Cl. A61B 19/00

(52) U.S. Cl. 606/130; 901/2

(57) ABSTRACT

It is an object of the present invention to provide an automated, a semi-automated, a surgeon-guided quasi-automated and/or a fully surgeon's controlled surgical system surgical system useful for performing a fully automated medical procedure within a body cavity such that faultless and quick medical procedure is obtained. Each of the surgical systems comprises: (a) at least one effecter for performing the medical procedure; (b) at least one maneuverable platform reversibly coupled with the effecter; the platform provides the effecter with a scheduled set of independent displacements selected from a group consisting of up to six (degrees of freedom) DOFs, namely linear movement along the X,Y,Z-coordinates, and radial movement around the X,Y,Z coordinates, such that the time-resolved spatial position of the effecter is defined by the up to six coordinates (three-dimensional spatial position, 3DSP); and (c) sensing and processing means.

### FIELD OF THE INVENTION

The present invention provides a robotic, i.e., an automated, a semi-automated, surgeon-guided quasi-automated and/or fully surgeon's controlled surgical system for obtaining or performing a quick and faultless medical procedure within a body cavity.

### BACKGROUND OF THE INVENTION

Medical errors are a major concern. In the United States medical errors are estimated to result in 44,000 to 98,000 unnecessary deaths and 1,000,000 unnecessary injuries each year (Institute of Medicine (2000) To Err is Human: “Building a safer health system” (2000), The National Academies Press; Charatan, Fred (2000), “Clinton acts to reduce medical mistakes”, BMJ Publishing Group). According to Leape L L (“Error in medicine”, JAMA 272 (23), 1851-7, 1994). It is estimated that in a typical 100 to 300 bed hospital in the United States, excess costs of $1,000,000 to $3,000,000 attributable to prolonged stays and complications due to medical errors occur yearly.

Yet still according to Leape L L, almost a quarter (22.7%) of active-care patient deaths were rated as at least possibly preventable by optimal care, with 6.0% rated as probably or definitely preventable.

Medical errors are usually caused by a combination of several factors. The most severe are complicated procedures, new procedures, inexperienced clinicians, complex care and urgent care.

One example of a complicated procedure is Transurethral Resection of the Prostate. As men age, their prostates can enlarge, leading to inability to urinate; bleeding through the urethra; kidney damage caused by urine backing up; frequent urinary tract infections; stones in the bladder.

The procedure of transurethral resection of the prostate takes about 60 minutes. During the procedure the surgeon inserts a resectoscope through the urethra and up to the prostate gland. A blade at the end of the scope is used to remove obstructing prostate tissue and then the blood vessels are sealed. Transurethral resection of the prostate is a very skill-demanding and time-consuming operation requiring many repetitive motions of the resectroscope. Therefore, up to 30% of men who undergo TURP experience many possible risks and complications associated with TURP, such as: bleeding in the urine (hematuria), infection, problems controlling urine flow (incontinence) and urethral stricture (tightening of the urethral outlet), difficulties achieving and maintaining erection (impotence), infertility, emptying of semen into the bladder instead of out of the urethra (dry climax).

Complication avoidance in all kinds of medical procedures is crucial since it will minimize patient morbidity and health care costs.

Current operative techniques rely on human surgeons, who have variable skill and dexterity. They also have physiological limits to their precision, tactile sensibility and stamina.

Surgical robots have the potential to increase the consistency and quality of medical procedures and offer dramatic improvements.

Traditional surgery relies on the physician's surgical skills and dexterity. Surgical robots have recently been developed to address the physical human issues such as fatigue and tremor in procedures. These systems were specifically developed for Minimally Invasive Surgery (MIS).

The Intuitive Surgical Inc. da Vinci and Computer Motion ZEUS robots are examples of MIS robots. U.S. Pat. No. 6,394,998 to Wallace et al issued relates to the da Vinci system. The da Vinci system according to U.S. Pat. No. 6,394,998 has seven degrees of freedom. The surgeon controls the robot through a console placed in the operating room, allowing control of both the external and internal surgical environments. The surgeon's interface has instrument controllers that can filter tremor and decrease the scale of motion. Foot pedals expand the surgeon's repertoire, allowing tissue coagulation and irrigation. Visual feedback is obtained through a proprietary stereoscopic display, called Surgical Immersion™.

Surgical robots in orthopaedics may be classified as positioning or machining aids. Robodoc, used for hip replacement surgery, is an example of the latter. Examples of patent literature which relate to these surgical robots are U.S. Pat. Nos. 5,695,500; 5,397,323 (both Taylor) U.S. Pat. No. 5,086,401; and U.S. Pat. No. 5,408,409 (both Glassman) issued in 1992 to 1997.

However none of those robots are able to control the entire performance of the physician during a medical procedure and to provide a quick and faultless medical procedure.

Thus, there is still a long felt need for an automated; a semi-automated; surgeon-guided quasi-automated; and/or a fully surgeon's controlled surgical system that will enable a quick and faultless medical procedure within a body cavity.

### SUMMARY OF THE INVENTION

It is one object of the present invention to provide an automated surgical system (100) useful for performing a fully automated medical procedure within a body cavity, such that a faultless and quick medical procedure is obtained. The system comprises effecters, maneuverable platform and sensing & processing means. It is in the scope of the invention wherein at least one effecter (10) performing the medical procedure is provided. Similarly, it is according to one embodiment of the invention wherein at least one maneuverable platform (20) reversibly coupled with the effecter (10) is provided. The platform provides the effecter with a scheduled set of independent displacements selected from a group consisting of up to six degrees of freedom (DOFs), e.g., 4 DOFs. Those DOFs are selected in a non-limiting manner from one or more of the group consisting of linear movement along the X, Y, Z-coordinates, and radial movement around the X,Y,Z coordinates. The time-resolved spatial position of the effecter is defined by the up to six coordinates (three-dimensional spatial position, 3DSP). The sensing and processing means (30) comprising means selected in a non limiting manner from (i) means (31) for acquiring a continuous real-time image of the body portion to be treated; (ii) means (32) for acquiring a continuous real-time 3DSP of said effecter; (iii) means (33) for processing the procedural displacement protocol; and (iv) means (34) for providing said at least one effecter and the maneuverable platform with the scheduled procedural displacement protocol.

It is another object of the present invention to provide a semi-automated surgical system (200) for obtaining a quick and faultless medical procedure within a body cavity. The system comprises, inter alia, modules as follows: (a) at least one effecter (10) performing the medical procedure; and, (b) at least one maneuverable platform (20) reversibly coupled with said effecter (10). The platform provides the effecter with independent displacements selected from a group consisting of up to six DOFs as defined above; and (c) sensing and processing means (30). This means comprises modules selected from a group of (i) means (31) for acquiring a continuous real-time image of the body portion to be treated; said body portion comprises a plurality n of (volumes of interest) VOIs having ALLOWED portions and NOT-ALLOWED portions; n is an integer number equal to or higher than 0; (ii) means (32) for acquiring a continuous real-time 3DSP of said effecter; and, (iii) means (35) for (a) actuating & positioning and/or (b) operating said at least one effecter solely within said ALLOWED portions.

It is another object of the present invention to provide a surgeon-guided quasi-automated surgical system (300) for obtaining a quick and faultless medical procedure within a body cavity. The system is (i) initially guided or programmed by the surgeon, and then (ii) either automatically or semi-automatically operated by the system; The system (300) comprises modules as follows: (a) at least one effecter (10) performing said medical procedure; (b) at least one maneuverable platform (20) reversibly coupled with said effecter (10); said platform provides said effecter with independent displacements selected from a group consisting of up to six DOFs as defined above and (c) sensing and processing means (30) for projecting first, second and third images. The means are selected from a group consisting of e.g., (i) means (31) for acquiring a continuous real-time first image of the body portion to be treated; (ii) means (36) for obtaining from said surgeon, who is manually operating said system (300) said second image comprising spatial positions of a plurality n of (volume of interest) VOIs having ALLOWED portions and NOT-ALLOWED portions; n is an integer number equal to or higher than 0; (iii) means (37) for acquiring a continuous real-time third image comprising 3DSP of said effecter; and, (iv) means (38) for superimposing said first, second and third images to enable (a) actuating & positioning and/or (b) operating said at least one effecter solely within said ALLOWED portions.

It is another object of the present invention to provide a fully surgeon's controlled surgical system (400) for obtaining a quick and faultless medical procedure within a body cavity. The system is fully regulated, restrained and monitored by said surgeon. The system comprises, inter alia, modules as follows: (a) at least one effecter (10) performing the medical procedure; and, (b) at least one maneuverable platform (20) reversibly coupled with said effecter (10). The platform provides the effecter with independent displacements selected from a group consisting of up to six DOFs as defined above; and (c) sensing and processing means (30). This means comprises modules selected from a group of (i) means (31) for acquiring a continuous real-time image of the body portion to be treated; said body portion comprises a plurality n of (volumes of interest) VOIs having ALLOWED portions and NOT-ALLOWED portions; n is an integer number equal to or higher than 0; (ii) means (32) for acquiring a continuous real-time 3DSP of said effecter; (iii) means (35) for (a) actuating & positioning and/or (b) operating said at least one effecter; and, (iv) means (39) for restricting said (a) actuating & positioning and/or (b) operating means of said at least one effecter solely within said ALLOWED portions.

It is another object of the present invention to provide the surgical system as defined above, wherein the sensing means are selected from a group consisting of AC or DC magnetic tracking Doppler, ultrasonic, RF, conductivity sensors, pressure sensors or any combination thereof. The sensing means are possibly targeted in parallel to the main longitudinal axis of the effecter.

It is another object of the present invention to provide the surgical system as defined above, wherein the effecter is selected from a group consisting of a maneuverable blade, wire, wire combined with diathermy system, coagulating member, vacuum device, laser, light emitting, radiation, heat or cold member, suturing mechanism, forceps, high pressure water injection, diagnostic means, ultrasound radiation, Doppler or any combination thereof.

It is another object of the present invention to provide the surgical system as defined above, wherein at least one effecter comprises an outer sheath accommodating a laser fiber, suitable for emitting a laser for either coagulation or resection of biological tissues; said outer sheath is an elongated member having a distal portion and a proximal portion. The distal proton is introduced within said body cavity, and the proximal portion is located outside the body.

It is another object of the present invention to provide the surgical system as defined above, additionally comprising a visualizing means intercommunicated with the sensing and processing means, the visualizing means adapted to provide the surgeon with a 3DSP of the effecter within the body cavity.

It is another object of the present invention to provide the surgical system as defined above, wherein the procedural displacement protocol is processed by a computer numerical control (CNC).

It is another object of the present invention to provide a fully automated method for performing a medical procedure within a body cavity, such that a faultless and quick medical procedure is obtained. The method comprises steps selected inter alia from the following: (a) obtaining at least one displaceable effecter (10) and a maneuverable platform; (b) at least partially reversibly coupling said platform with said effecter; (c) providing said effecter with a scheduled set of independent displacements selected from a group consisting of up to six DOFs, namely linear movement along the X,Y,Z-coordinates, and radial movement around the X,Y,Z coordinates; (d) defining the time-resolved spatial position of said effecter by the up to six coordinates (three-dimensional spatial position, 3DSP); (e) acquiring a continuous real-time image of the body portion to be treated; (f) acquiring a continuous real-time 3DSP of the effecter; (g) processing the procedural displacement protocol; (h) providing the at least one effecter and the maneuverable platform with the scheduled procedural displacement protocol; and, (i) performing the medical procedure by displacing and operating the at least one effecter (10) while maneuvering said at least one platform (20) according to the scheduled procedural displacement protocol; such that a faultless and quick medical procedure is obtained.

It is another object of the present invention to provide a semi-automated method for performing a medical procedure within a body cavity such that a faultless and quick medical procedure is obtained. The method comprises steps selected inter alia from: (a) obtaining at least one displaceable effecter (10) and a maneuverable platform; (b) at least partially reversibly coupling said platform with said effecter; (c) providing said effecter with a scheduled set of independent displacements selected from a group consisting of up to six DOFs, namely linear movement along the X,Y,Z-coordinates, and radial movement around the X,Y,Z coordinates; (d) defining the spatial position of said effecter by said up to six coordinates (three-dimensional spatial position, 3DSP); (e) acquiring a continuous real-time image of the body portion to be treated; said body portion comprises a plurality n of (volume of interest) VOIs having ALLOWED portions and NOT-ALLOWED portions; n is an integer number equal to or higher than 0; (f) acquiring a continuous real-time 3DSP of said effecter; (g) performing said medical procedure by (i) actuating & positioning and/or (ii) operating said at least one effecter (10) while maneuvering said at least one platform (20) solely within said ALLOWED portions; such that a faultless and quick medical procedure is obtained.

It is another object of the present invention to provide a surgeon-guided quasi-automated method for performing medical procedure within a body cavity such that a faultless and quick medical procedure is obtained. The method comprises steps selected inter alia from (a) obtaining at least one displaceable effecter (10) and a maneuverable platform; (b) at least partially reversibly coupling said platform with said effecter; (c) providing said effecter with a scheduled set of independent displacements selected from a group consisting of up to six DOFs, namely linear movement along the X,Y,Z-coordinates, and radial movement around the X,Y,Z coordinates; (d) defining the spatial position of said effecter by said up to six coordinates (three-dimensional spatial position, 3DSP); (e) obtaining a continuous real-time first image of the body portion to be treated; (f) obtaining from the surgeon, who is manually operating said system (300) said second image comprising spatial positions of a plurality n of (volume of interest) VOIs having ALLOWED portions and NOT-ALLOWED portions; n is an integer number equal to or higher than 0; (g) obtaining a continuous real-time third image comprising 3DSP of said effecter; (h) performing said medical procedure by superimposing said first, second and third images; thereby enabling (i) actuating & positioning and/or (ii) operating said at least one effecter (10) while maneuvering said at least one platform (20) solely within said ALLOWED portions; such that a faultless and quick medical procedure is obtained.

It is another object of the present invention to provide a fully surgeon's controlled method for performing medical procedure within a body cavity such that faultless and quick medical procedure is obtained. The method comprises steps selected inter alia from (a) obtaining at least one displaceable effecter (10) and a maneuverable platform; (b) at least reversibly coupling said platform with said effecter; (c) providing said effecter with a scheduled set of independent displacements selected from a group consisting of up to six DOFs, namely linear movement along the X,Y,Z-coordinates, and radial movement around the X,Y,Z coordinates; (d) defining the spatial position of said effecter by said up to six coordinates (three-dimensional spatial position, 3DSP); (e) acquiring a continuous real-time image of the body portion to be treated; said body portion comprises a plurality n of (volume of interest) VOIs having ALLOWED portions and NOT-ALLOWED portions; n is an integer number equal to or higher than 0; (f) acquiring a continuous real-time 3DSP of said effecter; and, (g) performing said medical procedure by (a) actuating & positioning and/or (b) operating said at least one effecter (10) whilst maneuvering and restricting said at least one platform (20) solely within said ALLOWED portions; such that a faultless and quick medical procedure is obtained.

It is still an another object of the present invention to provide the methods as defined above, additionally comprising the step of coupling to the effecter sensors selected from a group consisting of AC or DC magnetic tracking, Doppler, ultrasonic, RF, conductivity sensors, pressure sensors or any combination thereof.

It is lastly an object of the present invention to provide the methods as defined above, additionally comprising the step of processing said procedural displacement protocol by a computer numerical control (CNC).

### BRIEF DESCRIPTION OF THE DRAWINGS

The invention is herein described, by way of example only, with reference to the accompanying drawings, wherein:

FIG. 1 illustrates the fully automated surgical system (100).

FIG. 2 illustrates the semi-automated surgical system (200).

FIG. 3 illustrates the surgeon-guided quasi-automated surgical system (300).

FIG. 4 illustrates the fully surgeon's controlled surgical system (400).

FIG. 5-6 illustrate a general view of the system.

### DETAIL DESCRIPTION OF THE SPECIFIC EMBODIMENTS

The following description is provided, alongside all chapters of the present invention, so as to enable any person skilled in the art to make use of the invention and sets forth the best modes contemplated by the inventor of carrying out this invention. Various modifications, however, is adapted to remain apparent to those skilled in the art, since the generic principles of the present invention have been defined specifically to provide an automated, a semi-automated, surgeon-guided quasi-automated and/or a fully controlled surgical system for obtaining a quick and faultless medical procedure within a body cavity.

US application No. 2007/0078334 is incorporated in all its parts as a reference to the current invention.

The term “computer numerical control (CNC)” refers to a computer “controller” that reads G-code instructions and drives an effecter according to those instructions.

The term “programming language” refers herein after to an artificial language that can be used to control the behavior of a machine.

The term “G-code” refers to a programming language that controls the CNC.

The term “magnetic trackers” refers herein after to tracker or sensors used to capture the [x,y,z] translation coordinates and the rotation coordinates. There are two different types of magnetic trackers: direct current (DC) or alternating current (AC). Though they are in principal similar in operation, they differ in the manner in which they generate magnetic fields. The Field Generator coils are responsible for creating the magnetic fields and can be driven either by DC or AC. This means that the resultant magnetic fields are either constant (DC) or changing (AC).

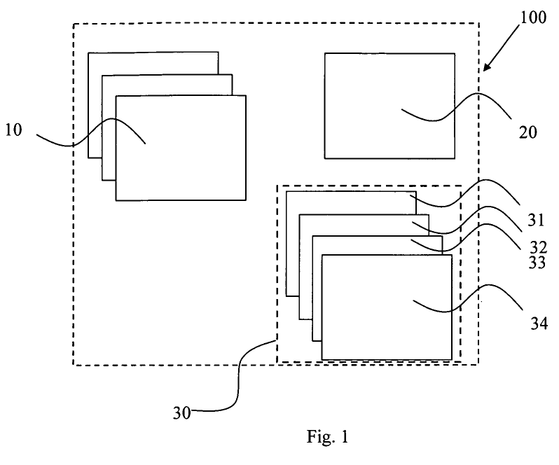

Reference is made now to FIG. 1, presenting in a non-limiting manner an automated surgical system (100) useful for performing a fully automated medical procedure within a body cavity, such that faultless and quick medical procedure is obtained. The automated surgical system (100) comprises: (a) at least one effecter (10) performing the medical procedure; (b) at least one maneuverable platform (20) reversibly coupled with the effecter (10). The platform provides the effecter with a scheduled set of independent displacements selected from a group consisting of up to six DOFs, namely linear movement along the X,Y,Z-coordinates, and radial movement around the X,Y,Z coordinates, such that the time-resolved spatial position of the effecter is defined by the up to six coordinates (three-dimensional spatial position, 3DSP). (c) sensing and processing means (30), comprising inter alia: means (31) for acquiring a continuous real-time image of the body portion to be treated; means (32) for acquiring a continuous real-time 3DSP of the effecter; means (33) for processing the procedural displacement protocol; and, means (34) for providing the at least one effecter and the maneuverable platform with the scheduled procedural displacement protocol.

This fully automated robotic system is especially adapted to provide a quick surgical procedure, where the role of the surgeon is minimized. The surgeon may thus possess less skills, expertise and experience, while a faultless procedure is still obtained. Both the medical repeatability and medical accuracy provided by system (100) are increased. The term “medical repeatability” refers hereinafter to the ability of a skilled or an unskilled physician to obtain the same or similar results in a series of medical procedures. The term “medical accuracy” refers hereinafter to the extent or degree to which results or outcome of a medical procedure approaches or matches the natural or optimal results.

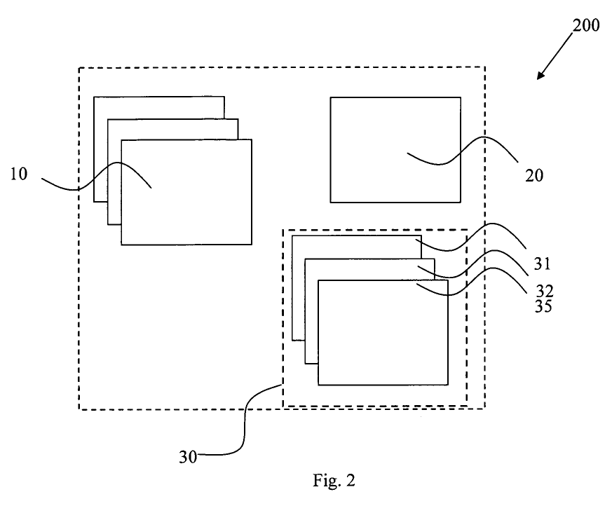

Reference is made now to FIG. 2, presenting in a non-limiting manner a semi-automated surgical system (200) for obtaining a quick and faultless medical procedure within a body cavity. The semi-automated surgical system (200) comprises (a) at least one effecter (10) for performing the medical procedure; (b) at least one maneuverable platform (20) partially reversibly coupled with the effecter (10). The platform provides the effecter with independent displacements selected from a group consisting of up to six DOFs, namely linear movement along the X, Y, Z-coordinates, and radial movement around the X, Y, Z coordinates, such that the spatial position of the effecter is defined by the up to six coordinates (three-dimensional spatial position, 3DSP); and (c) sensing and processing means (30). The sensing and processing means comprises means (31) for acquiring a continuous real-time image of the body portion to be treated; the body portion comprises a plurality n of (volume of interest) VOIs having ALLOWED portions and NOT-ALLOWED portions; n is an integer number equal to or higher than 0; means (32) for acquiring a continuous real-time 3DSP of the effecter; and, means (35) for (a) actuating & positioning and/or (b) operating the at least one effecter solely within the ALLOWED portions.

This semi-automated robotic system is operated in the following manner: (i) the surgeon introduces the effecter within the body of the patient; (ii) the surgeon places the effecter according to the surgical procedure, while the effecter is not operated; and while he/she marks the ALLOWED portions and/or NOT-ALLOWED portions of the VOIs. This mark is provided e.g., by inputting the system with an On or Off signal. The effecter, at this point, is possibly changed to a signaling probe rather then a real surgical tool. Simultaneously and subsequently, the sensing means acquires a real-time 3D image of the effecter and VOIs within the body. (iii) After displacing the effecter or probe as defined in step (ii), the effecter is displaced to the initial position; and, the robotic system operates the platform and effecter in an automatic manner.

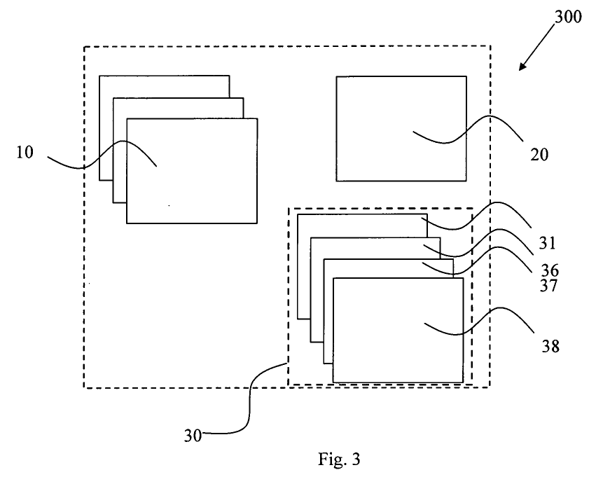

Reference is made now to FIG. 3, presenting in a non-limiting manner a surgeon-guided quasi-automated surgical system (300) for obtaining a quick and faultless medical procedure within a body cavity. The surgeon-guided quasi-automated surgical system (300) is (i) initially guided or programmed by the surgeon, and then (ii) either automatically or semi-automatically self-operated. The system (300) comprises (a) at least one effecter (10) performing the medical procedure; (b) at least one maneuverable platform (20) partially reversibly coupled with said effecter (10); said platform provides said effecter with independent displacements selected from a group consisting of up to six DOFs, namely linear movement along the X,Y,Z-coordinates, and radial movement around the X,Y,Z coordinates, such that the spatial position of said effecter is defined by said up to six coordinates (three-dimensional spatial position, 3DSP); and, (c) sensing and processing means (30) for projecting a first, second and third images. The sensing and processing means comprising means (31) for acquiring a continuous real-time first image of the body portion to be treated; means (36) for obtaining from said surgeon, who is manually operating said system (300) said second image comprising spatial positions of a plurality n of (volume of interest) VOIs having ALLOWED portions and NOT-ALLOWED portions; n is an integer number equal to or higher than 0; means (37) for acquiring a continuous real-time third image comprising 3DSP of said effecter; and, means (38) for superimposing said first, second and third images to enable (a) actuating & positioning and/or (b) operating said at least one effecter solely within said ALLOWED portions.

This quasi-automated robotic system is initially guided by the surgeon and only subsequently the system operates either fully automatically or semi-automatically. The quasi-automated system is adapted to perform in the following manner: (i) the surgeon introduces the effecter within the body of the patient; (ii) the surgeon provides the system with an image containing the spatial positions of the VOIs. Simultaneously and subsequently, the sensing means acquires a real-time 3D image of the effecter and VOIs within the body. After overlapping or superimposing the three images (iii) the effecter is displaced to the initial position; and, the robotic system maneuvers the platform and the effecter in an automatic manner to perform the medical procedure.

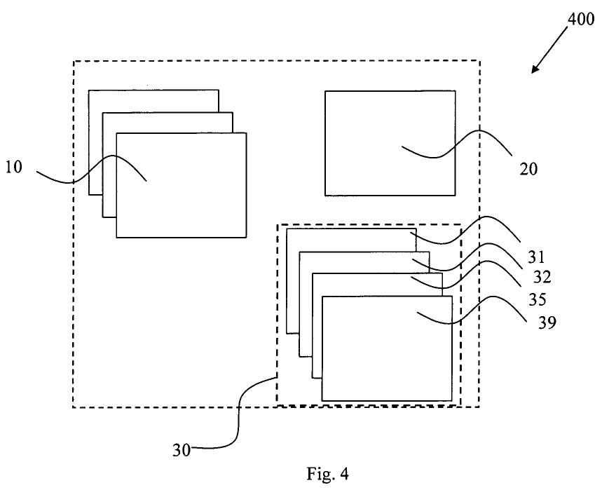

Reference is made now to FIG. 4, presenting in a non-limiting manner a fully surgeon's controlled surgical system (400) for obtaining a quick and faultless medical procedure within a body cavity. The fully surgeon's controlled surgical system (400) is fully regulated, restrained and monitored by the surgeon. The system (400) comprises (a) at least one effecter (10) performing the medical procedure; (b) at least one maneuverable platform (20) partially reversibly coupled with said effecter (10); said platform provides said effecter with independent displacements selected from a group consisting of up to six DOFs, namely linear movement along the X,Y,Z-coordinates, and radial movement around the X,Y,Z coordinates, such that the spatial position of said effecter is defined by said up to six coordinates (three-dimensional spatial position, 3DSP); and, (c) sensing and processing means (30). The sensing and processing means comprises means (31) for acquiring a continuous real-time image of the body portion to be treated; the body portion comprises a plurality n of (volume of interest) VOIs having ALLOWED portions and NOT-ALLOWED portions; n is an integer number equal to or higher than 0; means (32) for acquiring a continuous real-time 3DSP of the effecter; means (35) for (a) actuating & positioning and/or (b) operating the at least one effecter; and means (39) for restricting said (a) actuating & positioning and/or (b) operating means of said at least one effecter solely within said ALLOWED portions.

The fully surgeon's controlled surgical system (400) provides a quick and faultless medical procedure within a body cavity. The system (400) enables the use of a more powerful and intense effecters without endangering the patient. Hence, a much shorter procedure is obtained. The surgical system (400) follows/controls the surgeon's movements and prevents any medical fault (by actuating & positioning and/or operating the effecter within the NOT-ALLOWED portions of the VOIs). The system may vibrate the effecter so as to eliminate any medical fault.

According to one embodiment of the invention, the sensing and processing means within the robotic systems (either the fully automated surgical system 100; the semi-automated surgical system 200; the surgeon-guided quasi-automated surgical system 300; or the fully surgeon's controlled surgical system 400), as defined above, are utilized in a CNC, which reads G-code (artificial language that can be used to control the behavior of a machine) instructions and drives an effecter according to those instructions.

According to another embodiment of the present invention, the sensing means within the robotic systems (either the fully automated surgical system 100; the semi-automated surgical system 200; or the surgeon-guided quasi-automated surgical system 300; or the fully surgeon's controlled surgical system 400), as defined above, comprises miniature magnetic sensors that utilize 3D real-time imaging. The sensors may be selected from AC or DC magnetic tracking, Doppler, ultrasonic, RF, conductivity sensors, pressure sensors or any combination thereof.

According to yet another embodiment of the present invention, the effecter as defined in any of the robotic systems (either the fully automated surgical system 100; the semi-automated surgical system 200; or the surgeon-guided quasi-automated surgical system 300; or the fully surgeon's controlled surgical system 400) can be selected from a group consisting of a maneuverable blade, wire combined with and without diathermy system, coagulating member, vacuum device, laser, light emitting, radiation, heat or cold member, suturing mechanism, forceps, high pressure water injection diagnostic means, ultrasound radiation, Doppler or any combination thereof.

According to still another embodiment of the present invention, the fully automated surgical system 100; the semi-automated surgical system 200; or the surgeon-guided quasi-automated surgical system 300; or the fully surgeon's controlled surgical system 400 additionally comprises surgeon operated means especially adapted to enable the surgeon to start, stop, continue and restart the surgical system (either 100, 200, 300 or 400). The surgeon operated means can be selected from a group consisting of manual, foot or a microphone in communication with a computer, for receiving voice commands from the surgeon.

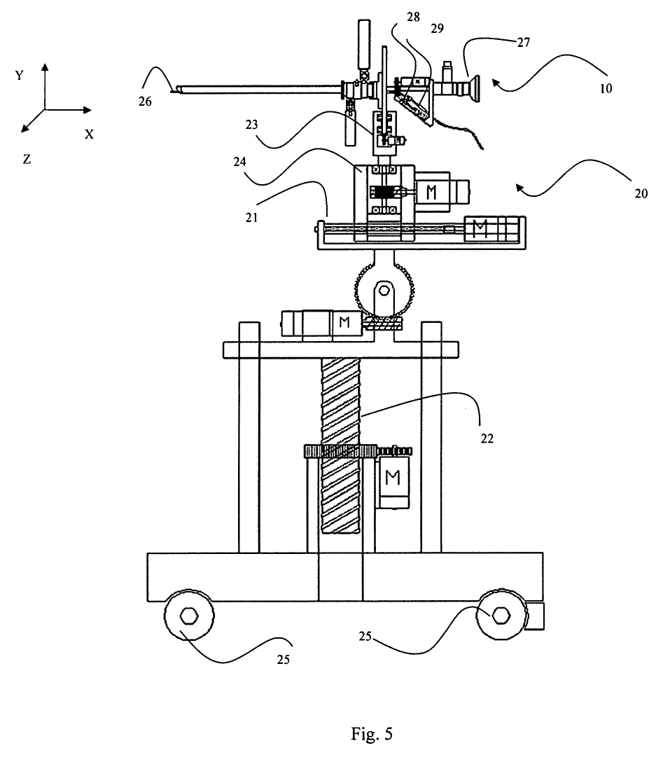

Reference is now made to FIG. 5 which represent a general view of the automated system (either the fully automated surgical system 100; the semi-automated surgical system 200; or the surgeon-guided quasi-automated surgical system 300; or the fully surgeon's controlled surgical system 400) according to a preferred embodiment of the present invention. The surgical system comprises (a) an effecter 10 adapted to perform a medical procedure within a body cavity and (b) a maneuverable platform 20.

The maneuverable platform 20 is adapted to be partially reversibly coupled to the effecter 10 and to provide the effecter (10) with independent displacements, namely linear movement along the X axis (21), linear movement along the Y axis (22), rotational movement around the X axis (23) and rotational movement around the Y axis (24).

The maneuverable platform 20 additionally comprises wheels (25) for enabling the displacement of the surgical system. Optionally the wheels have a locking mechanism for fixating the system.

The effecter (10) may be selected from a group consisting, preferably yet not solely, of a maneuverable blade, wire, coagulating member, vacuum device, laser, light emitting, radiation, suturing mechanism, forceps, diagnostic means, high water pressure injection or any combination thereof.

According to a preferred embodiment of the present invention the effecter comprises an outer sheath accommodating a laser fiber (26), suitable for emitting a laser for either coagulation or resection of biological tissues. The outer sheath is an elongated member having a distal portion introduced within the body cavity; and, a proximal portion, positioned outside said body.

According to yet another embodiment of the present invention, the surgical system additionally comprises sensing means, especially AC or DC magnetic tracking, utilizing real-time 3D imaging, Doppler, ultrasonic, RF, conductivity sensors, pressure sensors or any combination thereof. Those sensing means are preferably positioned along the main longitudinal axis of the effecter.

The system may additionally comprise an eye piece and camera connection (27) enabling the surgeon to obtain an image of the effecter and the body portion to be treated.

Furthermore, the system may comprise a mechanism adapted to permit the laser fiber with rotational movement (28) and/or linear movement (29).

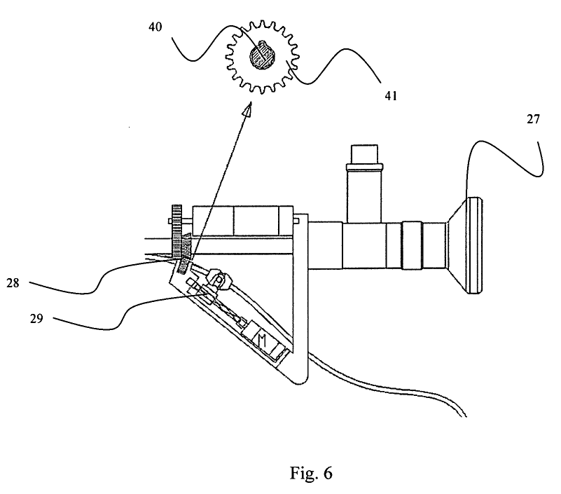

Reference is now made to FIG. 6 illustrating a closer view of the laser fiber's linear (29) and rotational (28) mechanisms. The rotational mechanism (28) includes at least two gears (40 and 41). The first gear (40) is adapted to accommodate the laser fiber (26, see FIG. 4) and to provide the laser (26) with linear movement but not rotary. The second gear (41) is adapted to actuate the rotary motion of the laser fiber (26, see FIG. 4).

1. An automated surgical system (100) useful for performing a fully automated medical procedure within a body cavity such that faultless and quick medical procedure is obtained; said system comprising:
a. at least one effecter (10) performing said medical procedure; and,
b. at least one maneuverable platform (20) reversibly coupled with said effecter (10); said platform provides said effecter with a scheduled set of independent displacements selected from a group consisting of up to six (degrees of freedom) DOFs, namely linear movement along the X,Y,Z-coordinates, and radial movement around the X,Y,Z coordinates, such that the time-resolved spatial position of said effecter is defined by said up to six coordinates (three-dimensional spatial position, 3DSP);
c. sensing and processing means (30), comprising
(i) means (31) for acquiring a continuous real-time image of the body portion to be treated;
(ii) means (32) for acquiring a continuous real-time 3DSP of said effecter;
(iii) means (33) for processing the procedural displacement protocol; and
(iv) means (34) for providing said at least one effecter and said maneuverable platform said scheduled procedural displacement protocol.

2. A semi-automated surgical system (200) for obtaining a quick and faultless medical procedure within a body cavity, said system comprising:
a. at least one effecter (10) performing said medical procedure; and,
b. at least one maneuverable platform (20) reversibly coupled with said effecter (10); said platform provides said effecter with independent displacements selected from a group consisting of up to six DOFs, namely linear movement along the X,Y,Z-coordinates, and radial movement around the X,Y,Z coordinates, such that the spatial position of said effecter is defined by said up to six coordinates (three-dimensional spatial position, 3DSP);
c. sensing and processing means (30), comprising:
(i) means (31) for acquiring a continuous real-time image of the body portion to be treated; said body portion comprises a plurality n of (volume of interest) VOIs having ALLOWED portions and NOT-ALLOWED portions; n is an integer number equal to or higher than 0;
(ii) means (32) for acquiring a continuous real-time 3DSP of said effecter; and,
(iii) means (35) for (a) actuating & positioning and/or (b) operating said at least one effecter solely within said ALLOWED portions.

3. A surgeon-guided quasi-automated surgical system (300) for obtaining a quick and faultless medical procedure within a body cavity, said system is (i) initially guided by said surgeon, and then (ii) either automatically or semi-automatically operated by said system; system (300) comprising:
a. at least one effecter (10) performing said medical procedure;
b. at least one maneuverable platform (20) reversibly coupled with said effecter (10); said platform provides said effecter with independent displacements selected from a group consisting of up to six DOFs, namely linear movement along the X,Y,Z-coordinates, and radial movement around the X,Y,Z coordinates, such that the spatial position of said effecter is defined by said up to six coordinates (three-dimensional spatial position, 3DSP); and,
c. sensing and processing means (30) for projecting a first, second and third images, said means comprising:
(i) means (31) for acquiring a continuous real-time first image of the body portion to be treated;
(ii) means (36) for obtaining from said surgeon, who is manually operating said system (300) said second image comprising spatial positions of a plurality n of (volume of interest) VOIs having ALLOWED portions and NOT-ALLOWED portions; n is an integer number equal to or higher than 0;
(iii) means (37) for acquiring a continuous real-time third image comprising 3DSP of said effecter; and,
(iv) means (38) to superimposing said first, second and third images to enable (a) actuating & positioning and/or (b) operating said at least one effecter solely within said ALLOWED portions.

4. A fully surgeon's controlled surgical system (400) for obtaining a quick and faultless medical procedure within a body cavity, said system is fully regulated, restrained and monitored by said surgeon, said system comprising:
a. at least one effecter (10) performing said medical procedure; and,
b. at least one maneuverable platform (20) reversibly coupled with said effecter (10); said platform provides said effecter with independent displacements selected from a group consisting of up to six DOFs, namely linear movement along the X,Y,Z-coordinates, and radial movement around the X,Y,Z coordinates, such that the spatial position of said effecter is defined by said up to six coordinates (three-dimensional spatial position, 3DSP);
c. sensing and processing means (30), comprising:
(i) means (31) for acquiring a continuous real-time image of the body portion to be treated; said body portion comprises a plurality n of (volume of interest) VOIs having ALLOWED portions and NOT-ALLOWED portions; n is an integer number equal to or higher than 0;
(ii) means (32) for acquiring a continuous real-time 3DSP of said effecter; and,
(iii) means (35) for (a) actuating & positioning and/or (b) operating said at least one effecter;
(iv) means (39) for restricting said (a) actuating & positioning and/or (b) operating means of said at least one effecter solely within said ALLOWED portions.

5-16. (canceled)

17. The surgical system according to claim 1, wherein said sensing means are selected from a group consisting of AC or DC magnetic tracking, Doppler, ultrasonic, RF, conductivity sensors, pressure sensors or any combination thereof.

18. The surgical system according to claim 2, wherein said sensing means are selected from a group consisting of AC or DC magnetic tracking, Doppler, ultrasonic, RF, conductivity sensors, pressure sensors or any combination thereof.

19. The surgical system according to claim 3, wherein said sensing means are selected from a group consisting of AC or DC magnetic tracking, Doppler, ultrasonic, RF, conductivity sensors, pressure sensors or any combination thereof.

20. The surgical system according to claim 4, wherein said sensing means are selected from a group consisting of AC or DC magnetic tracking, Doppler, ultrasonic, RF, conductivity sensors, pressure sensors or any combination thereof.

21. The surgical system according to claim 17, wherein said sensing means are targeted in parallel to the main longitudinal axis of the effecter.

22. The surgical system according to claim 18, wherein said sensing means are targeted in parallel to the main longitudinal axis of the effecter.

23. The surgical system according to claim 19, wherein said sensing means are targeted in parallel to the main longitudinal axis of the effecter.

24. The surgical system according to claim 20, wherein said sensing means are targeted in parallel to the main longitudinal axis of the effecter.

25. The surgical system according claim 1, wherein said effecter is selected from a group consisting of a maneuverable blade, wire combined with and without diathermy system, coagulating member, vacuum device, laser, light emitting, radiation, heat or cold member, suturing mechanism, forceps, high pressure water injection diagnostic means, ultrasound radiation, Doppler or any combination thereof.

26. The surgical system according claim 2, wherein said effecter is selected from a group consisting of a maneuverable blade, wire combined with and without diathermy system, coagulating member, vacuum device, laser, light emitting, radiation, heat or cold member, suturing mechanism, forceps, high pressure water injection diagnostic means, ultrasound radiation, Doppler or any combination thereof.

27. The surgical system according claim 3, wherein said effecter is selected from a group consisting of a maneuverable blade, wire combined with and without diathermy system, coagulating member, vacuum device, laser, light emitting, radiation, heat or cold member, suturing mechanism, forceps, high pressure water injection diagnostic means, ultrasound radiation, Doppler or any combination thereof.

28. The surgical system according claim 4, wherein said effecter is selected from a group consisting of a maneuverable blade, wire combined with and without diathermy system, coagulating member, vacuum device, laser, light emitting, radiation, heat or cold member, suturing mechanism, forceps, high pressure water injection diagnostic means, ultrasound radiation, Doppler or any combination thereof.

29. The surgical system according to claim 25, wherein at least one effecter comprises an outer sheath accommodating a laser fiber, suitable for emitting laser for either coagulation or resection of biological tissues; said outer sheath is an elongated member having a distal portion and a proximal portion, said distal proton is introduced within said body cavity, and said proximal portion locates outside said body.

30. The surgical system according to claim 26, wherein at least one effecter comprises an outer sheath accommodating a laser fiber, suitable for emitting laser for either coagulation or resection of biological tissues; said outer sheath is an elongated member having a distal portion and a proximal portion, said distal proton is introduced within said body cavity, and said proximal portion locates outside said body.

31. The surgical system according to claim 27, wherein at least one effecter comprises an outer sheath accommodating a laser fiber, suitable for emitting laser for either coagulation or resection of biological tissues; said outer sheath is an elongated member having a distal portion and a proximal portion, said distal proton is introduced within said body cavity, and said proximal portion locates outside said body.

32. The surgical system according to claim 16, wherein at least one effecter comprises an outer sheath accommodating a laser fiber, suitable for emitting laser for either coagulation or resection of biological tissues; said outer sheath is an elongated member having a distal portion and a proximal portion, said distal proton is introduced within said body cavity, and said proximal portion locates outside said body.

33. The surgical system according to claim 1, additionally comprising a visualizing means intercommunicated with said sensing and processing means, said visualizing means adapted to provide the surgeon with a 3DSP of said effecter within the body cavity.

34. The surgical system according to claim 2, additionally comprising a visualizing means intercommunicated with said sensing and processing means, said visualizing means adapted to provide the surgeon with a 3DSP of said effecter within the body cavity.

35. The surgical system according to claim 3, additionally comprising a visualizing means intercommunicated with said sensing and processing means, said visualizing means adapted to provide the surgeon with a 3DSP of said effecter within the body cavity.

36. The surgical system according to claim 4, additionally comprising a visualizing means intercommunicated with said sensing and processing means, said visualizing means adapted to provide the surgeon with a 3DSP of said effecter within the body cavity.

37. The surgical system according to claim 1, wherein said procedural displacement protocol is processed by a computer numerical control (CNC).

38. The surgical system according to claim 2, wherein said procedural displacement protocol is processed by a computer numerical control (CNC).

39. The surgical system according to claim 3, wherein said procedural displacement protocol is processed by a computer numerical control (CNC).

40. The surgical system according to claim 4, wherein said procedural displacement protocol is processed by a computer numerical control (CNC).

41. A fully automated method for performing medical procedure within a body cavity such that faultless and quick medical procedure is obtained, said method comprising steps of:
a. obtaining at least one displaceable effecter (10) and a maneuverable platform;
b. at least reversibly coupling said platform with said effecter;
c. providing said effecter with a scheduled set of independent displacements selected from a group consisting of up to six DOFs, namely linear movement along the X,Y,Z-coordinates, and radial movement around the X,Y,Z coordinates;
d. defining the time-resolved spatial position of said effecter by said up to six coordinates (three-dimensional spatial position, 3DSP);
e. acquiring a continuous real-time image of the body portion to be treated;
f. acquiring a continuous real-time 3DSP of said effecter;
g. processing the procedural displacement protocol;
h. providing said at least one effecter and said maneuverable platform said scheduled procedural displacement protocol; and,
i. performing said medical procedure by displacing and operating said at least one effecter (10) while maneuvering said at least one platform (20) according to said scheduled procedural displacement protocol; such that a faultless and quick medical procedure is obtained.

42. A semi-automated method for performing medical procedure within a body cavity such that faultless and quick medical procedure is obtained, said method comprising steps of:
a. obtaining at least one displaceable effecter (10) and a maneuverable platform;
b. at least reversibly coupling said platform with said effecter;
c. providing said effecter with a scheduled set of independent displacements selected from a group consisting of up to six DOFs, namely linear movement along the X,Y,Z-coordinates, and radial movement around the X,Y,Z coordinates;
d. defining the spatial position of said effecter by said up to six coordinates (three-dimensional spatial position, 3DSP);
e. acquiring a continuous real-time image of the body portion to be treated; said body portion comprises a plurality n of (volume of interest) VOIs having ALLOWED portions and NOT-ALLOWED portions; n is an integer number equal to or higher than 0;
f. acquiring a continuous real-time 3DSP of said effecter;
g. performing said medical procedure by (a) actuating & positioning and/or (b) operating said at least one effecter (10) while maneuvering said at least one platform (20) solely within said ALLOWED portions; such that a faultless and quick medical procedure is obtained.

43. A surgeon-guided quasi-automated method for performing medical procedure within a body cavity such that faultless and quick medical procedure is obtained, said method comprising steps of:
a. obtaining at least one displaceable effecter (10) and a maneuverable platform;
b. at least reversibly coupling said platform with said effecter;
c. providing said effecter with a scheduled set of independent displacements selected from a group consisting of up to six DOFs, namely linear movement along the X,Y,Z-coordinates, and radial movement around the X,Y,Z coordinates;
d. defining the spatial position of said effecter by said up to six coordinates (three-dimensional spatial position, 3DSP);
e. obtaining a continuous real-time first image of the body portion to be treated;
f. obtaining from the surgeon, who is manually operating said system (300) said second image comprising spatial positions of a plurality n of (volume of interest) VOIs having ALLOWED portions and NOT-ALLOWED portions; n is an integer number equal to or higher than 0;
g. obtaining a continuous real-time third image comprising 3DSP of said effecter;
h. performing said medical procedure by superimposing said first, second and third images; thereby enabling (a) actuating & positioning and/or (b) operating said at least one effecter (10) while maneuvering said at least one platform (20) solely within said ALLOWED portions; such that a faultless and quick medical procedure is obtained.

44. A fully surgeon's controlled method for performing medical procedure within a body cavity such that faultless and quick medical procedure is obtained, said method comprising steps of:
a. obtaining at least one displaceable effecter (10) and a maneuverable platform;
b. at least reversibly coupling said platform with said effecter;
c. providing said effecter with a scheduled set of independent displacements selected from a group consisting of up to six DOFs, namely linear movement along the X,Y,Z-coordinates, and radial movement around the X,Y,Z coordinates;
d. defining the spatial position of said effecter by said up to six coordinates (three-dimensional spatial position, 3DSP);
e. acquiring a continuous real-time image of the body portion to be treated; said body portion comprises a plurality n of (volume of interest) VOIs having ALLOWED portions and NOT-ALLOWED portions; n is an integer number equal to or higher than 0;
f. acquiring a continuous real-time 3DSP of said effecter;
g. performing said medical procedure by (a) actuating & positioning and/or (b) operating said at least one effecter (10) whilst maneuvering and restricting said at least one platform (20) solely within said ALLOWED portions; such that a faultless and quick medical procedure is obtained.

45. The methods according to claim 41, additionally comprising the step of coupling to said effecter sensors selected from a group consisting of AC or DC magnetic tracking.

46. The methods according to claim 42, additionally comprising the step of coupling to said effecter sensors selected from a group consisting of AC or DC magnetic tracking.

47. The methods according to claim 43, additionally comprising the step of coupling to said effecter sensors selected from a group consisting of AC or DC magnetic tracking.

48. The methods according to claim 44, additionally comprising the step of coupling to said effecter sensors selected from a group consisting of AC or DC magnetic tracking.

49. The method according to claim 42, additionally comprising the step of processing said procedural displacement protocol by a computer numerical control (CNC).
